(19) United States
(12) Patent Application Publication
(10) Pub. No.: US 2026/0106022 A1
(43) Pub. Date: Apr. 16, 2026

(54) ORCHESTRATION OF HEALTHCARE DATA COLLECTION

(71) Applicant: Savannah BRAYMILLER, Winterville, GA (US)

(72) Inventor: Savannah BRAYMILLER, Winterville, GA (US)

(21) Appl. No.: 19/353,938

(22) Filed: Oct. 9, 2025

Related U.S. Application Data
(60) Provisional application No. 63/705,629, filed on Oct. 10, 2024.

(51) Int. Cl. G16H 40/63; G06F 3/0482; G06F 3/0488

(52) U.S. Cl. CPC G16H 40/63; G06F 3/0482; G06F 3/0488

(57) ABSTRACT

Various implementations disclosed herein include devices, systems, and methods for implementing a healthcare data collection process. For example, a process may include, at a data collection device including one or more processors, a display, and a plurality of programmable input elements, detecting a first input from a first input element of the plurality of programmable input elements. The process may further include providing a first view of a data collection user interface on the display and obtaining user interaction data and health capture data associated with a patient for a healthcare session. The process may further include detecting a second input from a second input element of the plurality of programmable input elements. The process may further include analyzing the health capture data based on a criterion associated with the first input element and providing, for display via the data collection user interface, the analyzed health capture data.

### CROSS-REFERENCE TO RELATED APPLICATIONS

This application claims the benefit of U.S. Provisional Application Ser. No. 63/705,629 filed Oct. 10, 2024, each of which is incorporated by reference herein in its entirety.

### TECHNICAL FIELD

The present disclosure generally relates to systems, methods, and electronic devices for displaying analyzed health capture data for healthcare workers.

### BACKGROUND

Existing techniques for collecting healthcare data may not be adequate for efficiently and accurately collecting data while working with patients. For example, some healthcare workers, such as behavioral therapists, need to be able to have his or her hands available to provide effective patient care during a therapy session (e.g., working with a younger patient, such as a child). Thus, there is a need for improved techniques for a data collection device that may be specifically tailored for healthcare workers, and in particular, for healthcare workers, such as behavioral therapists, that need to collect data quickly and efficiently while working directly with patients.

### SUMMARY

Various implementations disclosed herein include devices, systems, and methods that provide a healthcare data collection process utilizing a unique wearable data collection device designed for healthcare workers while working directly with a patient (e.g., a doctor, nurse, behavioral therapist, etc.). In particular, the data collection device is designed for practitioners that need to collect data quickly and efficiently while working directly with patients (e.g., behavioral therapists). The healthcare data collection process may be implemented for observing patient behavior and acquiring what is known as “ABC data”. ABC data offers a framework for understanding behavior and is the foundation of Applied Behavior Analysis (ABA). The term “ABC” refers to the context of a behavioral event, describing events that occur before and after a behavior you want to learn more about, e.g., the antecedent (A), the behavior (B) and the consequence (C).

In some implementations, the data collection device includes a touch screen display and a plurality of programmable input elements (e.g., three input push buttons). The data collection device may be configured to be worn on a portion of an arm (e.g., a forearm) of a user (healthcare practitioner, therapist, etc.). The data collection device may include a data collection graphical user interface (GUI). The healthcare data collection process is provided for automatically capturing application screens of the GUI for data capture and documentation during a healthcare session (e.g., a behavior therapy session). For example, the GUI may provide treatment plans for each patient with 3 buttons on the upper right side of the screen that can be programable for the needs of the caregiver during any certain session.

In some implementations, the device may include a touch screen but provide no other functions other than to collect successful and unsuccessful targets acquired within a particular therapy session, in addition to tracking time, ABC (Antecedent, Behavior, Consequence) data, and logging incidents or permanent product recordings. In other words, the device is not meant to be software installed on a mobile cellular device, but is meant to be a unique wearable device, such as a digital interactive note pad, that is Health Insurance Portability and Accountability Act (HIPPA) compliant so that various therapy practitioners can report more reliable and accurate data when closing out sessions.

Some implementations assess physiological data and other user information to help improve a user experience (e.g., during a therapy session). In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user should have the ability to access and otherwise find out anything that the system has collected or determined about him or her. For example, session note documentation and submissions may be captured as a reflection of what each session entails for review by authorized stakeholders.

In general, one innovative aspect of the subject matter described in this specification can be embodied in methods that include the actions of, at a data collection device including one or more processors, a display, and a plurality of programmable input elements, detecting a first input from a first input element of the plurality of programmable input elements, in response to detecting the first input from the first input element, providing a first view of a data collection user interface on the display, obtaining user interaction data and health capture data associated with a patient for a healthcare session, detecting a second input from a second input element of the plurality of programmable input elements, in response to detecting the second input from the second input element, analyzing the health capture data based on a criterion associated with the first input element, and providing, for display via the data collection user interface, the analyzed health capture data.

These and other embodiments may each optionally include one or more of the following features.

In some aspects, the plurality of programmable input elements of the data collection device includes at least two programmable input buttons. In some aspects, the at least two programmable input buttons are positioned on an outside of a housing of the data collection device.

In some aspects, detecting the first input from the first input element of the plurality of programmable input elements is based on detecting a force pressed onto the first input element. In some aspects, the display includes a touchscreen corresponding to one or more interactable elements of the data collection user interface.

In some aspects, obtaining user interaction data and health capture data associated with the patient for the healthcare session includes receiving user input via one or more of the plurality of programmable input elements, receiving user input at the data collection user interface, collecting data via one or more sensors at the data collection device, or a combination thereof. In some aspects, the data collection device includes the one or more sensors. In some aspects, the one or more sensors include at least one of a light intensity camera, a depth camera, a microphone, and a motion sensor.

In some aspects, the first view of the data collection user interface includes a timed therapy session. In some aspects, the timed therapy session displayed on the data collection user interface includes one or more sub-options associated with the timed therapy session, wherein the one or more sub-options includes at least one of a stereotypic behavior option, a variable reinforcement option, and a pairing option.

In some aspects, the actions further include detecting a third input from a third input element of the plurality of programmable input elements, and in response to detecting the third input from the third input element, providing a third view of a data collection user interface on the display, wherein the third view includes a tacting program.

In some aspects, the analyzed health capture data includes at least one of a timed session, antecedent behavior consequence (ABC) data, logged incident data, and a video or audio recording. In some aspects, providing the analyzed health capture data for display via the second view of the data collection user interface includes the analyzed health capture data includes a graphical display of the health capture data.

In some aspects, the actions further include receiving input at the data collection user interface associated with ending the healthcare session, and in response to determining an end of the healthcare session automatically compiling the analyzed health capture data, and providing the compiled analyzed health capture data to another device.

In some aspects, the plurality of programmable input elements are customized based on a user setting. In some aspects, the data collection device includes a protective outer housing. In some aspects, the data collection device includes at least one secure strap configured to secure the device to an arm of a user. In some aspects, the data collection device is a wearable device.

In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein. In accordance with some implementations, a non-transitory computer readable storage medium has stored therein instructions, which, when executed by one or more processors of a device, cause the device to perform or cause performance of any of the methods described herein. In accordance with some implementations, a device includes: one or more processors, a non-transitory memory, and means for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates an exemplary electronic data collection device and user interface in a physical environment, the device being operated by a healthcare worker during a therapy session with a patient in accordance with some implementations.

FIGS. 2A and 2B illustrate the exemplary electronic data collection device and user interface of FIG. 1 during a behavior crisis portion of the therapy session with the patient in accordance with some implementations.

FIG. 3 illustrates the exemplary electronic data collection device and user interface of FIG. 1 during a tacting portion of the therapy session with the patient in accordance with some implementations.

FIG. 4 illustrates the exemplary electronic data collection device and user interface of FIG. 1 during the creation of a session by the healthcare worker after a therapy session with the patient in accordance with some implementations.

FIG. 5 illustrates an exemplary protective outer sleeve for the electronic data collection device of FIG. 1 in accordance with some implementations.

FIG. 6 is a flowchart illustrating a method for implementing a healthcare data collection process in accordance with some implementations.

FIG. 7 is a block diagram of an electronic device of in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects and/or variants do not include all of the specific details described herein. Moreover, well-known systems, methods, components, devices and circuits have not been described in exhaustive detail so as not to obscure more pertinent aspects of the example implementations described herein.

The technology in this patent application is related to systems and methods for implementing a healthcare data collection process utilizing a unique wearable data collection device designed for healthcare workers while working directly with a patient (e.g., a doctor, nurse, behavioral therapist, etc.). In particular, the data collection device is designed for practitioners that need to collect data quickly and efficiently while working directly with patients (e.g., behavioral therapists). The healthcare data collection process may be implemented for observing patient behavior and acquiring what is known as “ABC data”. ABC data offers a framework for understanding behavior and is the foundation of Applied Behavior Analysis (ABA). The term “ABC” refers to the context of a behavioral event, describing events that occur before and after a behavior you want to learn more about, e.g., the antecedent (A), the behavior (B) and the consequence (C).

The data collection device includes a touch screen display and a plurality of programmable input elements (e.g., three input push buttons). The data collection device may be configured to be worn on a portion of an arm (e.g., a forearm) of a user (healthcare practitioner, therapist, etc.). The data collection device may include a data collection graphical user interface (GUI). The healthcare data collection process is provided for automatically capturing application screens of the GUI for data capture and documentation during a healthcare session (e.g., a behavior therapy session). For example, the GUI may provide treatment plans for each patient with 3 buttons on the upper right side of the screen that can be programable for the needs of the caregiver during any certain session. The device may include a touch screen but provide no other functions other than to collect successful and unsuccessful targets acquired within a particular therapy session, in addition to tracking time, ABC (Antecedent, Behavior, Consequence) data, and logging incidents or permanent product recordings. In other words, the device is not meant to be software installed on a mobile cellular device, but is meant to be a unique wearable device, such as a digital interactive note pad, that is HIPPA compliant so that various therapy practitioners can report more reliable and accurate data when closing out sessions.

In some implementations, the GUI may provide a practitioner access to different tacting card bundles that can be used during session. For example, if a younger patient (e.g., a child) correctly/incorrectly identifies a tacting card a sound will emit from the device and the results will be recorded and accessible in the data collection results location in the device.

There are several advantages of utilizing this standalone data collection device (e.g., not an app on a user's cell phone). For example, the data collection device is purely for session use and practitioners will not be able to access social media or any communication avenues through this device. The data collection device brings multiple ABA therapy tools and data collection methods to one software program that is accessible to the user without the need to use a tablet or computer that would need to run multiple different programs back and forth. The data collection device is designed to cohesively aid the practitioner in running a therapy session without taking time away from the client to record reliable and accurate data. It is not meant to assist in billing or scheduling of any kind. Additionally, the data collection device eliminates the need to use pen and paper to document trial progress, alternatively it replaces the need to input the data into a laptop or tablet as well. The data collection device is discrete such that if at a park and taking data, it draws attention to the client if the practitioner is walking around with a laptop. The data collection device is intended to be HIPPA compliant in every aspect in that personal data of the client as well as program information is not viewable by the device by others apart from the practitioner themselves. Moreover, the data collection device can allow a practitioner to program the device to supply tools and data collection methods as needed per client such as what tacting cards need to be accessible, what task analyses need to be available, and so forth per the program requirements.

More specifically, this technology includes a healthcare data collection process that includes the actions of, at a data collection device including one or more processors, a display, and a plurality of programmable input elements (e.g., input buttons), detecting input from a first input element of the plurality of programmable input elements (e.g., user pushes button 1 or may press the button twice to initiate program). The actions may further include in response to detecting the first input from the first input element, providing a first view of a data collection user interface on the display (e.g., first default program for button 1—e.g., a timed therapy session). The actions may further include obtaining user interaction data and health capture data associated with a patient for a healthcare session (e.g., this may include user input at the touch screen, or automatic data collection via one or more sensors—microphone, camera, motion, etc., and during a therapy session there may be several sub-options initiated by pushing button 1 again or twice, or using the touch screen: stereotypic behavior option, variable reinforcement option, pairing option, etc.). The actions may further include detecting input from a second input element of the plurality of programmable input elements (e.g., second default program for button 2 to provide data collection results: user pushes button 2 (once or twice) to initiate ending the session and providing the results). The actions may further include analyzing the health capture data based on a criterion associated with the first input element (e.g., default program 1—a timed therapy session). The actions may further include providing, for display via the data collection user interface, the analyzed health capture data (e.g., graphical display of the results; the data can be sent to a second device (computer, server, etc.) for analysis or storage and/or sent to other programs on the market).

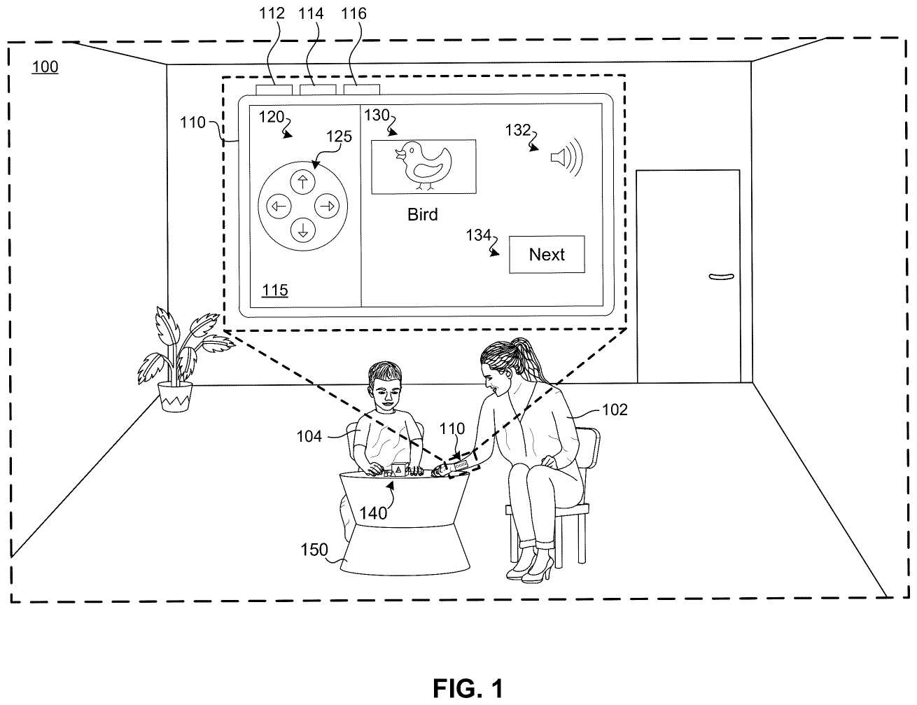

FIG. 1 illustrates an exemplary electronic device 110 operating in a physical environment 100. In this example of FIG. 1, the physical environment 100 is a room that includes a user 102, a patient 104, a table 150, while the patient 104 is interacting with some manipulatives 140 (e.g., toys or specific behavioral therapy toys). The user 102 (e.g., a healthcare practitioner, such as a behavioral therapist) is wearing the device 110, which may be affixed to his or her arm or wrist. The user 102 is conducting a behavioral therapy session (e.g., a healthcare session) with a patient 104 (e.g., a child).

FIG. 1 further illustrates an expanded view of a display of the device 110, including a screenshot of a data collection user interface 115. The device 110 may include one or more programmable input elements (e.g., programmable input elements 112, 114, 116). The programmable input elements 112, 114, 116 may each be customized to initiate a different application, of the one or more applications discussed herein. Additionally, or alternatively, different sequences of pushing each button may initiate a specific action for each different application (e.g., pushing programmable input element 116 twice (quickly) starts a clock, but pushing programmable input element 116 just once initiates a different application).

As illustrated in FIG. 1, the user 102 is conducting an initial healthcare session with the patient 104 as the patient is interacting with the behavioral therapy toys (e.g., manipulatives 140). The data collection user interface 115 provides a view of a timed therapy session application 120. The timed therapy session application 120 includes an interactable element 125 for providing controls to the user 102. Additionally, the timed therapy session application 120 includes interactable elements 130, 132, and 134 for providing a therapy session (e.g., having the patient identify sounds of a bird (or another animal), and providing the therapist with tools to enter notes and the like). During behavior therapy sessions, it may be imperative that accurate start and end times of sessions are documented without error, because each session is billable and to report incorrect times could hinder future opportunities for care.

In some implementations, the timed therapy session application 120 includes timer sub-options (for counts), and the programmable input elements 112, 114, 116 may be used to access particular functions (e.g., by pushing a button twice). The particular functions of the sub-options for the timed therapy session application 120 may include a stop watch, an alarm, identifying and tracking and stereotypic behaviors (e.g., this option will allow the user to time the duration of “stimming” such as hand flapping, rocking, or humming out of context), implementing a variable reinforcement option (e.g., this option will allow the user to keep count of trials so that when reinforcing behaviors in a fixed or variable schedule can be maintained, such as giving a sticker every five correct answers), and/or tracking the time spent specifically for pairing so that it may be documented. For example, a therapist (user 102) spent a total duration of 45 minutes pairing with the patient 104 to build rapport before starting trial activities (e.g., pairing is a provider-client bonding exercise that is continuous to build trust and increase instructional control).

In some implementations, the timed therapy session application 120 includes a data collection function and the programmable input elements 112, 114, 116 may be used to access particular functions (e.g., by pushing a button twice). The particular data collection functions of the timed therapy session application 120 may include tabs for different identified actions. For example, the different actions identified by the practitioner (user 102) may include motor imitation targets (e.g., Clap Hands: Number of Trials 13 Inputs +++−+−−++++++ This Session 10/13 or 77%; Touch Head: Number of Trials 5 Inputs +++++ This Session 5/5 or 100%), listener response targets (e.g., Make Eye Contact: NoT 20 Inputs++++++++++++++++++++ This Session 20/20 or 100%; Select the Cat: Number of Trials 4 Inputs−−−− This Session 0/4 or 0%), and/or identification of common things (e.g., Dog: Number of Trials 8 Inputs−−++++++ This Session 6/8 or 75%; Horse: Number of Trials 6 Inputs ++++++ This Session 6/6 or 100%). In some implementations, after accessing the data collection functions of the timed therapy session application 120 function, the user 102 may be able to scroll vertically to view all inputs and trial results in a categorized list so that similar targets are viewable together, even if run in the session a different order.

The electronic device 110 may include one or more sensors such as light intensity sensors (cameras), microphones, depth sensors, or other sensors that can be used to capture information about and evaluate the physical environment 100 and the objects within it, as well as information about the user 102 of the electronic device 110 and/or the patient 104. The information about the physical environment 100, user 102, and/or patient 104 may be used to provide visual and audio content associated with the healthcare session, and/or to identify the current location of the physical environment 100 and/or the location of the user within the physical environment 100. The electronic device 110 may include a speaker system to emit sound for timer functions or tacting (labeling) cards for correct answers or sound identifications. In some implementations, to protect personal information, a camera on the electronic device 110 would only be used to take pictures that did not disclose of any protected personal information during a therapy session.

People may sense or interact with a physical environment or world without using an electronic device. Physical features, such as a physical object or surface, may be included within a physical environment. For instance, a physical environment may correspond to a physical city having physical buildings, roads, and vehicles. People may directly sense or interact with a physical environment through various means, such as smell, sight, taste, hearing, and touch.

In some implementations, the device 110 obtains physiological data (e.g., EEG amplitude/frequency, pupil modulation, eye gaze saccades, etc.) from the user 102 and/or patient 104 via one or more sensors (e.g., a light intensity sensor such as a camera, a depth sensor, a motion sensor, and the like). For example, the device 110 obtains image data of the patient 104 during a therapy session from a camera and/or depth sensor. The device 110 may obtain motion data from a motion sensor (e.g., gyroscope, accelerometer, etc.) and/or other sources of physical environment information (e.g., camera positioning information from a camera's SLAM system, a visual inertial odometry (VIO) system, or the like). obtains pupillary data (e.g., eye gaze characteristic data). While this example and other examples discussed herein illustrates a single device 110 in a real-world physical environment 100, the techniques disclosed herein are applicable to multiple devices and multiple sensors, as well as to other real-world environments/experiences. For example, the functions of the device 110 may be performed by multiple devices.

In some implementations, the device 110 provides a digital screen meant to reflect the treatment plans for each patient with buttons on the upper right side of the screen that can be programable for the needs of the caregiver during any certain session. In some implementations, the digital screen is a touch screen and has no other functions than to collect successful and unsuccessful targets ran within a session, in addition to tracking time, ABC (Antecedent, Behavior, Consequence) data, and logging incidents or permanent product recordings during a healthcare session. In some implementations, the device 110 is not meant to be used as a tool to aid in parent training. In some implementations, the device 110 is meant to be a wearable and customizable (e.g., programmable) digital note pad that is HIPPA compliant so that various therapy practitioners can report more reliable and accurate data when closing out sessions.

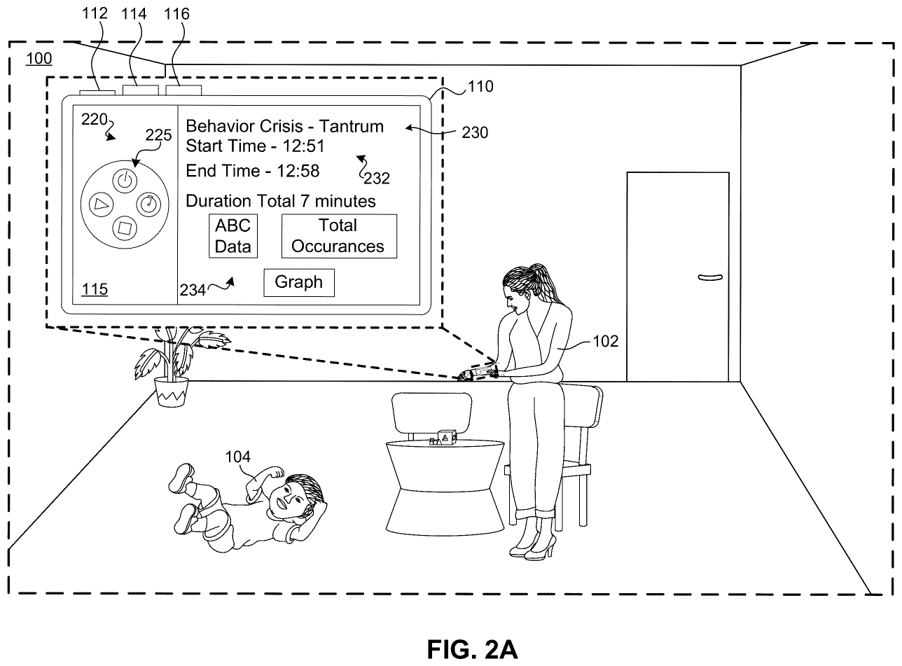

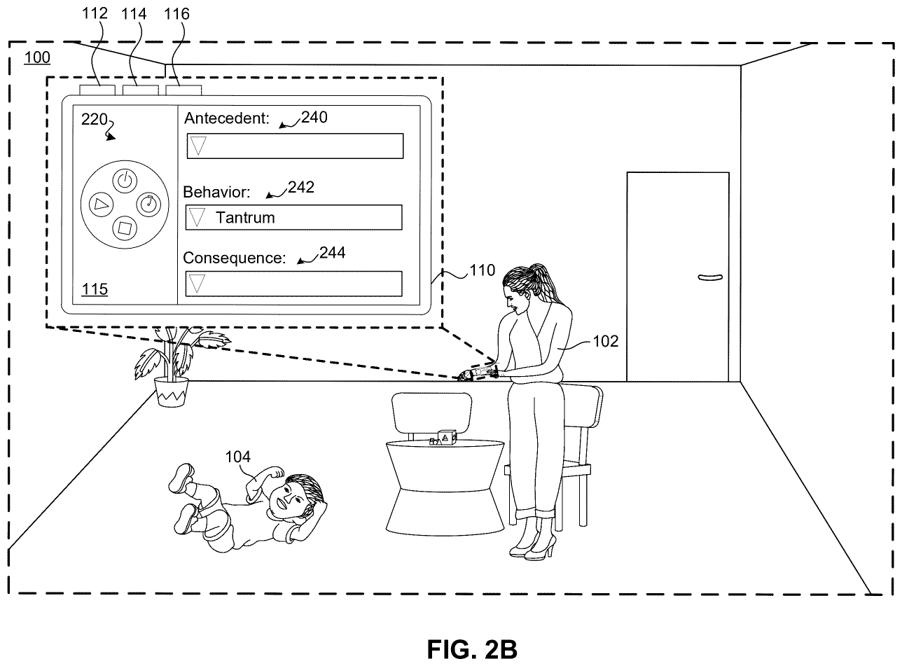

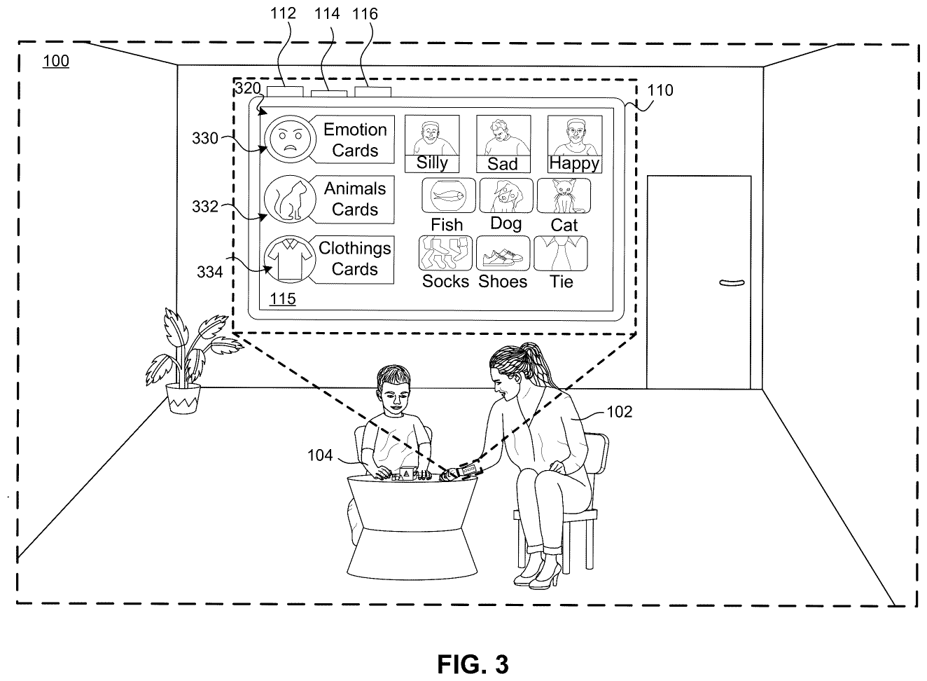

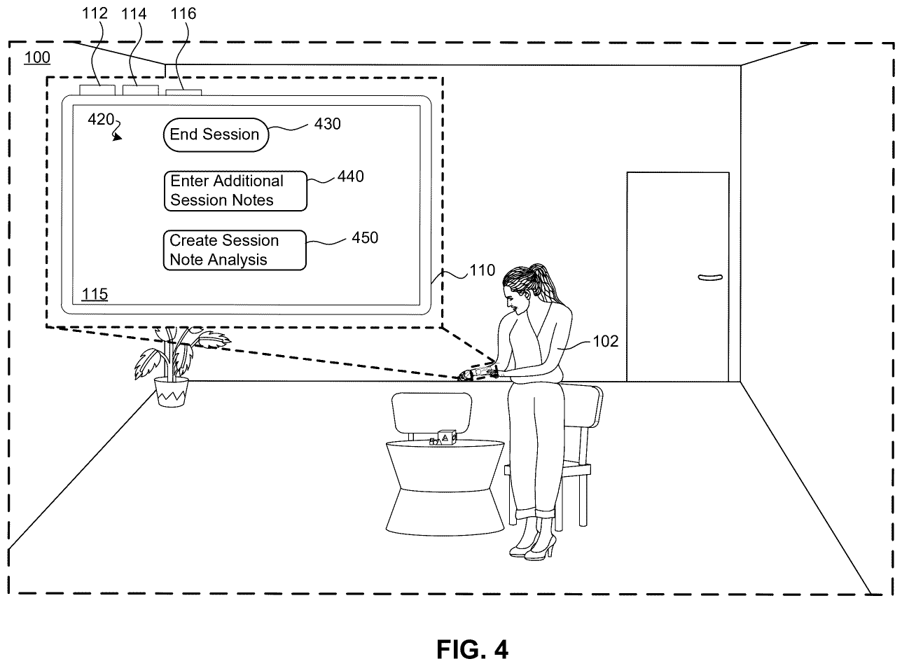

FIGS. 2A, 2B, 3, and 4 illustrate different portions of the healthcare session of FIG. 1 while the user 102 interacts with the device 110. For example, FIGS. 2A and 2B illustrate a behavior crisis (e.g., a temper tantrum), FIG. 3 illustrates a tacting portion of the healthcare session, and FIG. 4 illustrates entering session notes after the patient 104 has left the environment 100 (e.g., an end of a therapy session).

FIGS. 2A and 2B illustrate the exemplary electronic data collection device and user interface of FIG. 1 (e.g., data collection user interface 115) during a behavior crisis portion of the therapy session with the patient in accordance with some implementations. In particular, FIG. 2A illustrates the user 102 initiating a behavior crisis application 220 by selecting (e.g., pushing) the programmable input element 112 (e.g., pressing the top left button to initiate an app). The behavior crisis application 220 includes an interactable element 225 for providing controls to the user 102 (e.g., interactable element such as a timer, start, stop, and similar elements). The behavior crisis application 220 includes the element 230 for labeling the current session (e.g., a “tantrum”). The behavior crisis application 220 further includes the element 232 for displaying a timer. The behavior crisis application 220 further includes the elements at area 234, such as, inter alia, links to other programs such as ABC collection process, total occurrences, a graph to display current or past results for the patient, and the like.

FIG. 2B illustrates a different portion of the behavior crisis application 220 for entering ABC data from the current therapy session (e.g., after the user selected the ABC element at area 234. For example, the user 102 may enter different types of notes or select a category or note from a list from the one or more drop down elements, such as antecedent (“A”) at element 240, behavior (“B”) at element 242, and consequence (“C”) at element 244.

In some implementations, by selecting the graph element at area 234, the behavior crisis application 220 may initiate a graph function. The graph function may provide the user 102 with a visualization of a graph after stopping the session timer to view trends by clicking on the category of target they wish to view once the data points have been gathered. The user 102, via a graph, may be able to identify the trends to visualize progress and report to supervisor such as reporting percentage growth in session notes. For example, a baseline may start at 0% as the skill is not yet acquired, intervention is the progression of the goal until it is mastered, and maintenance is maintaining the skill after mastered.

FIG. 3 illustrates the exemplary electronic data collection device and user interface of FIG. 1 (e.g., data collection user interface 115) during a tacting portion of the therapy session with the patient in accordance with some implementations. In particular, FIG. 3 illustrates the user 102 initiating a tacting application 320 by selecting (e.g., pushing) the programmable input element 114 (e.g., pressing the middle button to initiate an app). For example, tacting may be a targeted behavior of acknowledging a stimuli and its presence in the environment, e.g., a chair in a room is just a chair, but if someone looks at it respectively and can identify it as a chair, the individual has performed a true tact. Thus, behavior therapists may use cards to teach clients what daily objects are and their respective functions and how to discriminate them from things like couches or stools, all of which you can sit.

In an exemplary implementation, the tacting application 320 provide a practitioner access to different tacting card bundles that can be used during session. For example, if a younger patient (e.g., a child) correctly/incorrectly identifies a tacting card a sound will emit from the device and the results will be recorded and accessible in the data collection results location in the device. For example, as illustrated in FIG. 3, the tacting application 320 includes emotion cards 330 (e.g., silly, sad, happy, and the like), animal cards 332 (e.g., fish, dog, cat, and the like), and clothing cards 334 (e.g., socks, shoes, tie, and the like). The customization of the data collection user interface 115 allows a practitioner (e.g., user 102) to program the device 110 to supply tools and data collection methods as needed per client such as what tacting cards need to be accessible, what task analyses need to be available, and so forth per the program or healthcare session requirements.

FIG. 4 illustrates the exemplary electronic data collection device and user interface of FIG. 1 (e.g., data collection user interface 115) during the creation of a session by the healthcare worker after a therapy session with the patient in accordance with some implementations. In particular, FIG. 4 illustrates the user 102 initiating a session creation application 420 by selecting (e.g., pushing) the programmable input element 116 (e.g., pressing the right button to initiate an app). The session creation application 420 provides the user the ability to add session notes and other information for the system to collect after the patient 104 has left the room (e.g., physical environment 100). For example, as illustrated in FIG. 4, the session creation application 420 includes the element 430 for ending a session, element 440 for allowing the user 102 enter additional session notes, and element 450 for initiating a create session note analysis (e.g., In an exemplary embodiment, the session creation application 420 allows the practitioner to create applied behavior analysis (ABA) session notes. ABA session notes are detailed records of therapy sessions between a client and a therapist or behavior analyst. They are essential for tracking a client's progress, planning future interventions, and ensuring consistent therapy delivery. Session notes can also help ABA professionals communicate with others, such as doctors and teachers, and can be of interest to anyone involved in the patient's care, including parents and insurance payors.

In an exemplary embodiment, two examples of a session note that may be automatically generated by the data collection user interface 115 are as follows:
1. The patient transitioned from their guardians to the therapist at 10:00 AM in the absence of maladaptive behaviors and they appeared content by finishing their fruit loops snack at the table in their therapy room at the clinic. The priority of the session was pairing in that the therapist is novel to the client. The reinforcement used throughout the session was social praise and access to an edible tangible consisting of chocolate chips or fruit snack gummies. Data for this session includes but is not limited to the following:
a. ECHOICS: The client will echo the word “headphones” within the first 5 seconds of presentation from the therapist. 100% accuracy out of 10 trials
b. LISTENER RESPONDING: The client will bring an item to the therapist within 8 seconds of presentation of demand from the therapist. 80% accuracy out of 5 trials
   Additionally, the client engaged in the behavior of biting when playing with blocks closely to a peer. Based on this observation, follow up with the BCBA is advised. The client transitioned to their guardians from the therapist in the absence of maladaptive behaviors during departure from the clinic at 1:00 PM. Data was collected in (data collection device for healthcare workers) and it is recommended that ABA therapy continue per the plan of care.
2. The patient transitioned from the AM therapist to the PM therapist at the clinic at 1:00 PM exhibiting problem behaviors including falling to the ground and crying with visible tears for a duration of 4 minutes and 32 seconds. The client appeared to be tired by rubbing their facing and using their ACC device to tell the therapist they wanted to go home and take a nap. The therapist responded by redirecting the client to engage in a preferred activity which consisted of being taken on a wagon ride throughout the clinic. The client persistently appeared tired throughout the session by rubbing their face and closing their eyes during breaks between discrete trial training tasks. The RBT observed that the client's cheeks were blushing and felt warm to the touch. In this, the client's temperature was taken by the supervising BCBA at 1:55 PM and the temperature reading displayed 101. The caregivers on file were notified immediately and the client left for departure at 2:13 PM. The following data collected throughout the session includes the following:
a. Total count of tantrum behaviors—3 instances
b. Total count of noncompliance—6 instances
c. ECHOICS: The client will repeat the word “green” within 4 seconds of presentation from the therapist. 50% accuracy out of 8 trials
   Data was collected in (data collection device for healthcare workers) and it is recommended that ABA therapy continue per the plan of care.

In some implementations, the data collection user interface 115 of the data collection device a task analysis feature may be initialized following a completed session. For example, a practitioner may have access to various task analysis targets in line with the program for a patient. A task analysis is a break down of steps to teach necessary life skills to the client such as brushing teeth. The practitioner may also be able to collect data on the steps done properly throughout the session without prompt in which results can be found in the data collection results page.

In some implementations, the data collection user interface 115 of the data collection device 110 may include additional data recording and session tools. For example, a practitioner may have access to a permanent product recording tool (e.g., a final recording of the end result of a behavior count: Throwing books on the floor PPR 13 books thrown on the floor after tantrum behavior), a token board (e.g., visual for the practitioner and client in which reinforcement consists of earning tokens that can be traded in for a reward), a social praise reference list (e.g., practitioners are encouraged to use varied social praise, because “good job” looses its ring after saying it to a client so many times it no longer acts as a reinforcer, varying social praise by said other things like “stupendous” or “you're terrific” aids in reinforcement, and a reference list for alternative social praises to use throughout session) ABC data for behaviors not relative to a crisis (e.g., not all ABC data needing to be taken is in result of crisis behavior), environmental factors tool (e.g., if the client is in the middle of a move with their family and has no toys to play with during session because they are packed away, this could cause a negative effect on the therapy session which can be notated here), a time sampling tool, an interval recording tool, and/or a latency recording.

Moreover, behavior therapists may need access to behavior intervention plans (BIP0 which is a strategy to be implemented in the event of a crisis. Something to note is that each case is client specific, so there is not a “one size fits all” approach to ABA therapy. In an exemplary implementation, the device 110 may be programed by the responsible party to best suit the needs of each practitioner specific to the needs of each client individually, in which where one client may need a BIP, another client may not. This may be applicable to all programing possibilities for the device 110, so to list all possible treatment plan interventions is not attainable. Thus, in an exemplary implementation, the device 110 may be programable from a factory default setting where responsible parties can change device specifications as needed per client.

The examples described herein for FIGS. 1-4 are non-limiting examples of some of the implementations for using the data collection user interface 115 of the data collection device 110. In various implementations, the data collection device 110 is designed for healthcare session use and practitioners may be prevented from accessing social media or any communication avenues through this device 110. The data collection device 110 brings multiple ABA therapy tools and data collection methods to one software program (data collection user interface 115) that is accessible to the user without the need to use a tablet or computer that would need to run multiple different programs back and forth. The data collection device 110 is designed to cohesively aid the practitioner in running a therapy session without taking time away from the client to record reliable and accurate data. The data collection device 110 is not meant to assist in billing or scheduling of any kind, but in some implementations, other applications may be used with the device 110. The data collection device 110 may eliminate a need to use pen and paper to document trial progress. The data collection device 110 may replace the need to input the data into a laptop or tablet as well. The data collection device 110 may be discrete such that if at a park and taking data, it draws attention to the client if the practitioner is walking around with a laptop. The data collection device 110 is intended to be HIPPA compliant in every aspect in that personal data of the client as well as program information is not viewable by the device by others apart from the practitioner themselves.

In some implementations, the practitioner can program the data collection device 110 to supply tools and data collection methods as needed per client such as what tacting cards need to be accessible, what task analyses need to be available, and so forth per the program requirements. For example, to customize the data collection device 110 to the needs of each practitioner, there may be computer software that is accessible by another device (e.g., a computer/laptop, a phone, tablet, etc.) to input goals, targets, and supplemental resources like tacting card bundles that will be synced to the device. Once a session is started, the data collection will begin. Once the session ends, the practitioner may close the session on the device 110 after gathering whatever data is needed for their session notes. At this time, the data may be erased and may no longer be accessed to remain HIPPA compliant. The practitioner may then need to start the session over for the next session or change over to a new saved programed session for another client (patient) that was put together in the same software. The goal is to make the device 110 compatible with other data collection software so that it can be paired with the other programs. In this case, the creator of the behavior intervention plan can adjust the device 110 specifications as needed per program.

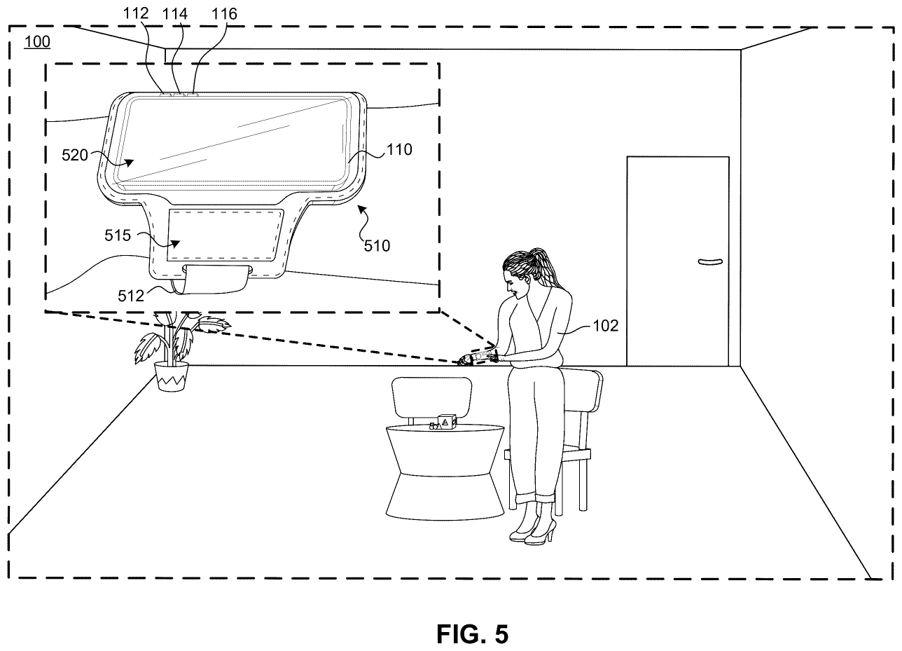

FIG. 5 illustrates an exemplary protective outer sleeve 510 for the electronic data collection device of FIG. 1 in accordance with some implementations. In particular, the protective outer sleeve 510 may store the device 110 within an enclosure (e.g., a secure housing). In an exemplary implementation, the outer sleeve 510 may include a strap 512 for securing the outer sleeve 510 to the user 102 and a clear overlay 520 that is touch screen compatible.

In some implementations, the protective outer sleeve 510 may include thin foam padding surrounding the outer edge surfaces of the device 110. For example, a primary function of thin foam padding surrounding the outer edge surfaces of the device 110 is to protect clients (e.g., patient 104) should they come into contact with the therapist (e.g., user 102) including but not limited to by running by the therapist or throwing themselves during a crisis. Additionally, or alternatively, in some implementations, the outer sleeve 510 may include a mesh like material such that the worn device 110 is not abrasive on the wearers skin in the event that sessions sometimes run for multiple hours.

In some implementations, a note section 515 of the outer sleeve 510 may be provided. For example, the note section 515 may provide a secure location that a therapist (e.g., user 102) may be able to store written notes or other thin materials that may be used during a therapy session.

Additionally, or alternatively, in some implementations, other types of fasteners may be used with, or in addition to, the secure strap 512 for strapping the device 110 to the wrist or another portion of the user or an object such that hard buttons or clips are not a factor in jeopardizing client safety. For example, other types of fasteners that may be used for securing the device 110 may include self-fasteners, refastenable tape, hook and loop fasteners/closures, double-sided adhesive tape, and the like.

In some implementations, the outer sleeve 510 includes a clear overlay 520 that is touch screen compatible. For example, the clear overlay 520 may be utilized such that the outer sleeve 510 can be sanitized to prevent the spread of illness between clients and to keep the inside device safe from bodily fluids such as spit or throw up in the event that a therapist come into contact with it in addition to other mediums that could harm the device 110 through natural environment teaching, direct trial training, or during times of patient crisis. In some implementations, the clear overlay may include a covering for the programmable input elements 112, 114, 116.

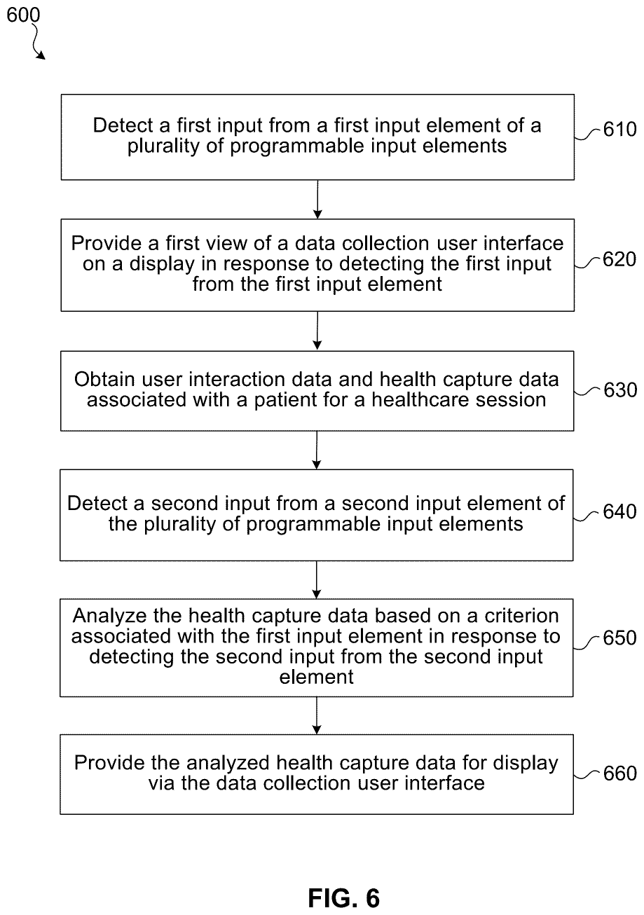

FIG. 6 is a flowchart illustrating a method 600 for implementing a healthcare data collection process in accordance with some implementations. In some implementations, a device such as electronic device 110 performs method 600. In some implementations, method 600 is performed on a mobile device, desktop, laptop, HMD (e.g., device 110), or server device. The method 600 is performed by processing logic, including hardware, firmware, software, or a combination thereof. In some implementations, the method 600 is performed on a processor executing code stored in a non-transitory computer-readable medium (e.g., a memory). In some implementations, the device performing the method 600 includes a processor, a display, a plurality of programmable input elements, and one or more sensors. In an exemplary embodiment, the method 600 is performed at a data collection device (e.g., device 110) that includes one or more processors, a display, and a plurality of programmable input elements (e.g., input buttons).

At block 610, the method 600 detects a first input from a first input element of the plurality of programmable input elements. For example, a health practitioner pushes a button or may press the button twice to initiate program. For example, as illustrated in FIG. 2A, the user 102 initiates a behavior crisis application 220 on the device 110 that he or she is wearing on their wrist/arm by selecting (e.g., pushing) the programmable input element 112 (e.g., pressing the top left button to initiate an app).

In some implementations, the plurality of programmable input elements of the data collection device includes at least two programmable input buttons. In some implementations, the at least two programmable input buttons are positioned on an outside of a housing of the data collection device.

At block 620, the method 600 provides a first view of a data collection user interface on the display in response to detecting the first input from the first input element. For example, as illustrated in FIG. 2A, the behavior crisis application 220 is initiated on the data collection user interface 115 at the device 110. In some implementations, the first view of the data collection user interface includes a timed therapy session. In some implementations, the timed therapy session displayed on the data collection user interface includes one or more sub-options associated with the timed therapy session, wherein the one or more sub-options includes at least one of a stereotypic behavior option, a variable reinforcement option, and a pairing option.

In some implementations, detecting the first input from the first input element of the plurality of programmable input elements is based on detecting a force pressed onto the first input element. For example, as illustrated in FIG. 2A, the user 102 initiates a behavior crisis application 220 on the device 110 that he or she is wearing on their wrist/arm by selecting (e.g., pushing) the programmable input element 112 (e.g., pressing the top left button to initiate an app).

At block 630, the method 600 obtains user interaction data and health capture data associated with a patient for a healthcare session. For example, this may include user input at the touch screen, or automatic data collection via one or more sensors, such as a microphone, camera, motion, etc. In some instances, during the therapy/healthcare session, there may be several sub-options initiated by pushing a button (e.g., programmable input elements 112, 114, 116) again or twice, or using the touch screen to initiate a stereotypic behavior option, a variable reinforcement option, a pairing option, and the like.

In some implementations, obtaining user interaction data and health capture data associated with the patient for the healthcare session includes receiving user input via one or more of the plurality of programmable input elements, receiving user input at the data collection user interface, collecting data via one or more sensors at the data collection device, or a combination thereof. In some implementations, the data collection device includes the one or more sensors. In some implementations, the one or more sensors includes at least one of a light intensity camera, a depth camera, a microphone, and a motion sensor (e.g., an IMU, a gyroscope, an accelerometer, etc.).

At block 640, the method 600 detects a second input from a second input element of the plurality of programmable input elements. For example, a default program for a button to end the session and provide data collection results: user pushes a button (e.g., programmable input element 116), either once or twice to initiate ending the session and providing the session results, add notes, and the like.

At block 650, the method 600 analyzing the health capture data based on a criterion associated with the first input element in response to detecting the second input from the second input element. For example, after the user 102 selected the end session element 430 and then create session note analysis 450, the system (via data collection user interface 115) analyzes the collected data for the particular healthcare session of the patient 104.

At block 660, the method 600 provides the analyzed health capture data for display via the data collection user interface. For example, the data collection user interface 115 may display a graphical analysis of the session and/or the data collection user interface 115 may automatically generate session notes as described herein for FIG. 4.

In some implementations, the method 600 further includes detecting a third input from a third input element of the plurality of programmable input elements, and in response to detecting the third input from the third input element, providing a third view of a data collection user interface on the display, wherein the third view includes a tacting program. For example, FIG. 3 illustrates the user 102 initiating a tacting application 320 by selecting (e.g., pushing) the programmable input element 114 (e.g., pressing the middle button to initiate an app). The tacting application 320 provide a practitioner access to different tacting card bundles that can be used during session. For example, if a younger patient (e.g., a child) correctly/incorrectly identifies a tacting card a sound will emit from the device and the results will be recorded and accessible in the data collection results location in the device. For example, as illustrated in FIG. 3, the tacting application 320 includes emotion cards 330 (e.g., silly, sad, happy, and the like), animal cards 332 (e.g., fish, dog, cat, and the like), and clothing cards 334 (e.g., socks, shoes, tie, and the like). The customization of the data collection user interface 115 allows a practitioner (e.g., user 102) to program the device 110 to supply tools and data collection methods as needed per client such as what tacting cards need to be accessible, what task analyses need to be available, and so forth per the program or healthcare session requirements.

In some implementations, the display includes a touchscreen corresponding to one or more interactable elements of the data collection user interface. In some implementations, the analyzed health capture data includes at least one of a timed session, antecedent behavior consequence (ABC) data, logged incident data, and a video or audio recording. In some implementations, providing the analyzed health capture data for display via the second view of the data collection user interface includes the analyzed health capture data includes a graphical display of the health capture data.

In some implementations, the plurality of programmable input elements are customized based on a user setting. For example, each programmable input elements (e.g., programmable input elements 112, 114, 116), and/or other interactable elements on the touchscreen display may be customized and programmed for different practitioners (e.g., different settings per user, such as, inter alia, creating shortcuts or hot buttons).

In some implementations, the method 600 further includes receiving input at the data collection user interface associated with ending the healthcare session, and in response to determining an end of the healthcare session automatically compiling the analyzed health capture data, and providing the compiled analyzed health capture data to another device. For example, compatible and analyzed data can be collected and automatically sent to another device (e.g., another mobile device, a computer, a remote server, cloud computing network, etc.) for compiling and/or analysis, such as to a main healthcare system computer network.

In some implementations, the data collection device 110 includes a protective outer housing. Additionally, or alternatively, in some implementations, the data collection device 110 includes at least one secure strap configured to secure the device to an arm of a user. For example, making sure that the data collection device 110 cannot danger a patient 104 or a user 102 in the event the patient 104 comes in contact with the device 110 during a crisis (e.g., a tantrum), or if the patient 104 runs past and bumps into the therapist. For example, the device 110 may be held by sleeves or straps with a soft outer material that securely encases the screen to prevent potential accidents.

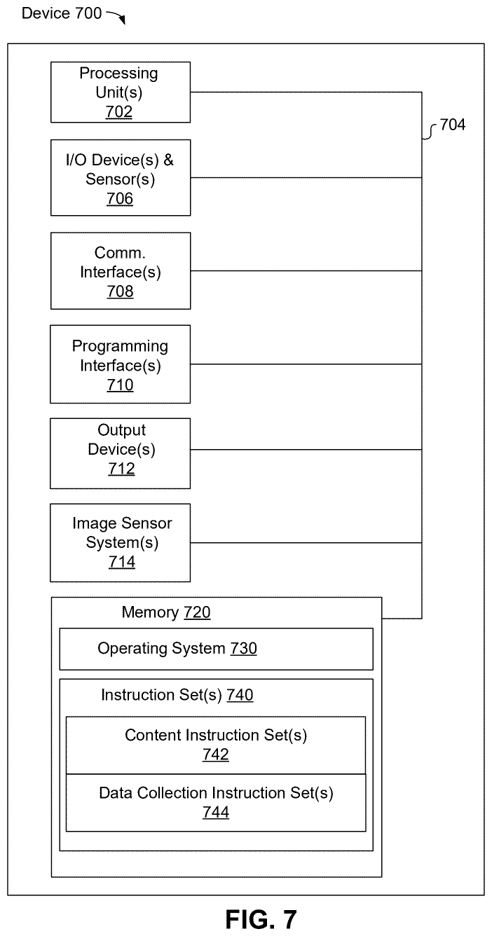

FIG. 7 is a block diagram of electronic device 700. Device 700 illustrates an exemplary device configuration for electronic device 110. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 700 includes one or more processing units 702 (e.g., microprocessors, ASICs, FPGAs, GPUs, CPUs, processing cores, and/or the like), one or more input/output (I/O) devices and sensors 706, one or more communication interfaces 708 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, I2C, and/or the like type interface), one or more programming (e.g., I/O) interfaces 710, one or more output device(s) 712, one or more interior and/or exterior facing image sensor systems 714, a memory 720, and one or more communication buses 704 for interconnecting these and various other components.

In some implementations, the one or more communication buses 704 include circuitry that interconnects and controls communications between system components. In some implementations, the one or more I/O devices and sensors 706 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more output device(s) 712 include one or more displays configured to present a view of a 3D environment to the user. In some implementations, the one or more device(s) 712 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays correspond to diffractive, reflective, polarized, holographic, etc. waveguide displays. In one example, the device 700 includes a single display. In another example, the device 700 includes a display for each eye of the user.

In some implementations, the one or more output device(s) 712 include one or more audio producing devices. In some implementations, the one or more output device(s) 712 include one or more speakers, surround sound speakers, speaker-arrays, or headphones that are used to produce spatialized sound, e.g., 3D audio effects. Such devices may virtually place sound sources in a 3D environment, including behind, above, or below one or more listeners. Generating spatialized sound may involve transforming sound waves (e.g., using head-related transfer function (HRTF), reverberation, or cancellation techniques) to mimic natural soundwaves (including reflections from walls and floors), which emanate from one or more points in a 3D environment. Spatialized sound may trick the listener's brain into interpreting sounds as if the sounds occurred at the point(s) in the 3D environment (e.g., from one or more particular sound sources) even though the actual sounds may be produced by speakers in other locations. The one or more output device(s) 712 may additionally or alternatively be configured to generate haptics.

In some implementations, the one or more image sensor systems 714 are configured to obtain image data that corresponds to at least a portion of a physical environment. For example, the one or more image sensor systems 714 may include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 714 further include illumination sources that emit light, such as a flash. In various implementations, the one or more image sensor systems 714 further include an on-camera image signal processor (ISP) configured to execute a plurality of processing operations on the image data.

The memory 720 includes high-speed random-access memory, such as DRAM, SRAM, DDR RAM, or other random-access solid-state memory devices. In some implementations, the memory 720 includes non-volatile memory, such as one or more magnetic disk storage devices, optical disk storage devices, flash memory devices, or other non-volatile solid-state storage devices. The memory 720 optionally includes one or more storage devices remotely located from the one or more processing units 702. The memory 720 includes a non-transitory computer readable storage medium.

In some implementations, the memory 720 or the non-transitory computer readable storage medium of the memory 720 stores an optional operating system 730 and one or more instruction set(s) 740. The operating system 730 includes procedures for handling various basic system services and for performing hardware dependent tasks. In some implementations, the instruction set(s) 740 include executable software defined by binary information stored in the form of an electrical charge. In some implementations, the instruction set(s) 740 are software that is executable by the one or more processing units 702 to carry out one or more of the techniques described herein.

The instruction set(s) 740 includes a content instruction set 742, and a data collection instruction set 744. The instruction set(s) 740 may be embodied a single software executable or multiple software executables.

In some implementations, the content instruction set 742 is executable by the processing unit(s) 702 to provide and/or track content for display on a device. The content instruction set 742 may be configured to monitor and track content over time (e.g., during an experience, i.e., a therapy session), as well as provide content for the user interface. In some implementations, the content instruction set 742 may be configured to provide. To these ends, in various implementations, the instruction includes instructions and/or logic therefor, and heuristics and metadata therefor.

In some implementations, the data collection instruction set 744 is executable by the processing unit(s) 702 to implement healthcare data collection processes described herein, utilizing a wearable data collection device designed for healthcare workers while working directly with a patient (e.g., a doctor, nurse, behavioral therapist, etc.). To these ends, in various implementations, the instruction includes instructions and/or logic therefor, and heuristics and metadata therefor.

Although the instruction set(s) 740 are shown as residing on a single device, it should be understood that in other implementations, any combination of the elements may be located in separate computing devices. Moreover, the figure is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

It will be appreciated that the implementations described above are cited by way of example, and that the present invention is not limited to what has been particularly shown and described hereinabove. Rather, the scope includes both combinations and sub combinations of the various features described hereinabove, as well as variations and modifications thereof which would occur to persons skilled in the art upon reading the foregoing description and which are not disclosed in the prior art.

As described above, one aspect of the present technology is the gathering and use of sensor data that may include user data to improve a user's experience of an electronic device. The present disclosure contemplates that in some instances, this gathered data may include personal information data that uniquely identifies a specific person or can be used to identify interests, traits, or tendencies of a specific person. Such personal information data can include movement data, physiological data, demographic data, location-based data, telephone numbers, email addresses, home addresses, device characteristics of personal devices, or any other personal information.

The present disclosure recognizes that the use of such personal information data, in the present technology, can be used to the benefit of users. For example, the personal information data can be used to improve the content viewing experience. Accordingly, use of such personal information data may enable calculated control of the electronic device. Further, other uses for personal information data that benefit the user are also contemplated by the present disclosure.

The present disclosure further contemplates that the entities responsible for the collection, analysis, disclosure, transfer, storage, or other use of such personal information and/or physiological data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates implementations in which users selectively block the use of, or access to, personal information data. That is, the present disclosure contemplates that hardware or software elements can be provided to prevent or block access to such personal information data. For example, in the case of user-tailored content delivery services, the present technology can be configured to allow users to select to “opt in” or “opt out” of participation in the collection of personal information data during registration for services. In another example, users can select not to provide personal information data for targeted content delivery services. In yet another example, users can select to not provide personal information, but permit the transfer of anonymous information for the purpose of improving the functioning of the device.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences or settings based on non-personal information data or a bare minimum amount of personal information, such as the content being requested by the device associated with a user, other non-personal information available to the content delivery services, or publicly available information.

In some embodiments, data is stored using a public/private key system that only allows the owner of the data to decrypt the stored data. In some other implementations, the data may be stored anonymously (e.g., without identifying and/or personal information about the user, such as a legal name, username, time and location data, or the like). In this way, other users, hackers, or third parties cannot determine the identity of the user associated with the stored data. In some implementations, a user may access their stored data from a user device that is different than the one used to upload the stored data. In these instances, the user may be required to provide login credentials to access their stored data.

Numerous specific details are set forth herein to provide a thorough understanding of the claimed subject matter. However, those skilled in the art will understand that the claimed subject matter may be practiced without these specific details. In other instances, methods apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general-purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

What is claimed is:

1. A method comprising:
at a data collection device comprising one or more processors, a display, and a plurality of programmable input elements:
detecting a first input from a first input element of the plurality of programmable input elements;
in response to detecting the first input from the first input element, providing a first view of a data collection user interface on the display;
obtaining user interaction data and health capture data associated with a patient for a healthcare session;
detecting a second input from a second input element of the plurality of programmable input elements;
in response to detecting the second input from the second input element, analyzing the health capture data based on a criterion associated with the first input element; and
providing, for display via the data collection user interface, the analyzed health capture data.

2. The method of claim 1, wherein the plurality of programmable input elements of the data collection device comprises at least two programmable input buttons.

3. The method of claim 2, wherein the at least two programmable input buttons are positioned on an outside of a housing of the data collection device.

4. The method of claim 1, wherein detecting the first input from the first input element of the plurality of programmable input elements is based on detecting a force pressed onto the first input element.

5. The method of claim 1, wherein the display comprises a touchscreen corresponding to one or more interactable elements of the data collection user interface.

6. The method of claim 1, wherein obtaining user interaction data and health capture data associated with the patient for the healthcare session comprises receiving user input via one or more of the plurality of programmable input elements, receiving user input at the data collection user interface, collecting data via one or more sensors at the data collection device, or a combination thereof.

7. The method of claim 6, wherein the data collection device comprises the one or more sensors.

8. The method of claim 6, wherein the one or more sensors comprise at least one of:
a light intensity camera;
a depth camera;
a microphone; and
a motion sensor.

9. The method of claim 1, wherein the first view of the data collection user interface comprises a timed therapy session.

10. The method of claim 9, wherein the timed therapy session displayed on the data collection user interface comprises one or more sub-options associated with the timed therapy session, wherein the one or more sub-options comprises at least one of a stereotypic behavior option, a variable reinforcement option, and a pairing option.

11. The method of claim 1, further comprising:
detecting a third input from a third input element of the plurality of programmable input elements; and
in response to detecting the third input from the third input element, providing a third view of a data collection user interface on the display, wherein the third view comprises a tacting program.

12. The method of claim 1, wherein the analyzed health capture data comprises at least one of a timed session, antecedent behavior consequence (ABC) data, logged incident data, and a video or audio recording.

13. The method of claim 1, wherein providing the analyzed health capture data for display via the data collection user interface comprises a graphical display of the health capture data.

14. The method of claim 1, wherein the plurality of programmable input elements are customized based on a user setting.

15. The method of claim 1, further comprising:
receiving input at the data collection user interface associated with ending the healthcare session; and
in response to determining an end of the healthcare session:
automatically compiling the analyzed health capture data, and
providing the compiled analyzed health capture data to another device.

16. The method of claim 1, wherein the data collection device comprises a protective outer housing.

17. The method of claim 1, wherein the data collection device comprises at least one secure strap configured to secure the device to an arm of a user.

18. The method of claim 1, wherein the data collection device is a wearable device.

19. A device comprising:
a display;
a plurality of programmable input elements;
a non-transitory computer-readable storage medium; and
one or more processors coupled to the non-transitory computer-readable storage medium, wherein the non-transitory computer-readable storage medium comprises program instructions that, when executed on the one or more processors, cause the one or more processors to perform operations comprising:
detecting a first input from a first input element of the plurality of programmable input elements;
in response to detecting the first input from the first input element, providing a first view of a data collection user interface on the display;
obtaining user interaction data and health capture data associated with a patient for a healthcare session;
detecting a second input from a second input element of the plurality of programmable input elements;
in response to detecting the second input from the second input element, analyzing the health capture data based on a criterion associated with the first input element; and
providing, for display via the data collection user interface, the analyzed health capture data.

20. A non-transitory computer-readable storage medium, storing program instructions executable on a device comprising one or more processors to perform operations comprising:
detecting a first input from a first input element of a plurality of programmable input elements;
in response to detecting the first input from the first input element, providing a first view of a data collection user interface on a display;
obtaining user interaction data and health capture data associated with a patient for a healthcare session;
detecting a second input from a second input element of the plurality of programmable input elements;
in response to detecting the second input from the second input element, analyzing the health capture data based on a criterion associated with the first input element; and
providing, for display via the data collection user interface, the analyzed health capture data.
